(19) United States
(12) Patent Application Publication
(10) Pub. No.: US 2021/0256647 A1
(43) Pub. Date: Aug. 19, 2021

(54) FOOD MONITORING ASSEMBLY AND METHOD

(71) Applicant: Carrier Corporation, Palm Beach Gardens, FL (US)

(72) Inventors: Preeti K. Mishra, Hyderabad (IN); Chakravarty Anumachpalli, Hyderabad (IN); Rahul Kankane, Hyderabad (IN)

(21) Appl. No.: 17/253,327

(22) PCT Filed: Dec. 17, 2019
(86) PCT No.: PCT/US2019/066838

§ 371 Date: Dec. 17, 2020

(30) Foreign Application Priority Data
Dec. 24, 2018 (IN) 201811048838

(51) Int. Cl. G06Q 50/28; G06K 7/10; G06K 19/07; G06Q 30/00; G05B 15/02; F25D 29/00; G01N 21/25; G01N 33/02

(52) U.S. Cl. CPC G06Q 50/28; G06K 7/10297; G06K 19/0723; G06Q 30/018; G05B 15/02; F25D 2700/08; G01N 21/251; G01N 33/02; G06K 2007/10504; F25D 2700/06; F25D 29/006

(57) ABSTRACT

A food monitoring assembly includes at least one color sensor (36) for measuring a color of a food product (30). A RFID tag reader (42) is configured to identify a RFID tag (40) in the vicinity of the RFID tag reader. A controller (50) is in electrical communication with the color sensor (36) and the RFID tag reader (42) and is configured to identify a type of the food product and determine if the color of the food product is within a predetermined color range. A communicator (58) is in electrical communication with the controller (50) for communication with a remote location.

### CROSS-REFERENCE TO RELATED APPLICATIONS

This application claims priority to Indian Provisional Application No. 201811048838, which was filed on Dec. 24, 2018 and is incorporated herein by reference.

### BACKGROUND

This application relates to a food monitoring assemblies and methods, particularly related to monitoring food condition and extending shelf life.

Food waste is a significant problem with approximately thirty percent of food in the United States spoiling between production and consumption. Spoiled food may also need to be separated from non-spoiled food to prevent further spoilage. Therefore, there is a need to reduce food waste and identify food once it has spoiled.

### SUMMARY

In one exemplary embodiment, a food monitoring assembly includes at least one color sensor for measuring a color of a food product. A RFID tag reader is configured to identify a RFID tag in the vicinity of the RFID tag reader. A controller is in electrical communication with the color sensor and the RFID tag reader and is configured to identify a type of the food product and determine if the color of the food product is within a predetermined color range. A communicator is in electrical communication with the controller for communication with a remote location.

In a further embodiment of any of the above, at least one biosensor is in electrical communication with the controller.

In a further embodiment of any of the above, at least one biosensor is configured to measure gas emissions emitted from the food product.

In a further embodiment of any of the above, at least one biosensor is configured to measure a pesticide level associated with the food product.

In a further embodiment of any of the above, at least one biosensor includes at least one chemical sensor that has a reactive bio-component element, a sensor element, and an interface element disposed there between.

In a further embodiment of any of the above, at least one solar cell is in electrical communication with a battery for powering the assembly.

In a further embodiment of any of the above, the color sensor is configured to measure a wave length of light to determine the color of the food product.

In a further embodiment of any of the above, the food monitor assembly is located adjacent a refrigeration cooler.

In a further embodiment of any of the above, the food monitor assembly is located within a food transport trailer.

In a further embodiment of any of the above, the controller includes a microprocessor and memory for storing food product data.

In a further embodiment of any of the above, the communicator includes a wireless connection for communicating with the remote location.

In another exemplary embodiment, a method of monitoring a condition of a food product includes the step of identifying a type of the food product with a RFID tag reader. A current color of the food product is measured with at least one color sensor. It's determined if the current color is within a predetermined range of colors for the food product. A user is signaled if the current color is outside of the predetermined range of colors for the food product.

In a further embodiment of any of the above, a pesticide level associated with the food product is measured using at least one biosensor.

In a further embodiment of any of the above, a user is signaled if the pesticide level is outside of a predetermined pesticide level range.

In a further embodiment of any of the above, the step of signaling to the user includes illuminating a light.

In a further embodiment of any of the above, measuring a gas emissions level associated with the food product using the at least one biosensor.

In a further embodiment of any of the above, the user is signaled if the gas emissions level is outside of a predetermined gas emissions range.

In a further embodiment of any of the above, the current color of the food product is compared with a previously measured color of the food product to determine a color change of the food product.

In a further embodiment of any of the above, a refrigerated system is adjusted to reduce food spoilage if the color change of the food product exceeds a predetermined level.

In a further embodiment of any of the above, food product data for the food product is accessed after the type of food product has been identified.

### BRIEF DESCRIPTION OF THE DRAWINGS

FIG. 1 illustrates an example refrigerated cooler including a food monitor.

FIG. 2 illustrates an enlarged view of the food monitor in the refrigerated cooler adjacent a food product.

FIG. 3 illustrates an example refrigerated container having multiple compartments each with at least one food monitor.

FIG. 4 illustrates an example method of monitoring a condition of the food product.

### DETAILED DESCRIPTION

FIG. 1 illustrates a refrigerated cooler 20 for storing a plurality of food products 30. In the illustrated example, the plurality of food products 30 can include produce, seafood, and/or dairy products. The refrigerated cooler 20 includes a cooler housing 22 that supports shelves 24 for displaying the food products 30 to customers, such as in a supermarket. The refrigerated cooler 20 also includes a refrigeration system 26 integrated into the cooler housing 22 to provide conditioned air to each of the plurality of shelves 24 through vents 28 in fluid communication with the refrigeration system 26 to extend a shelf life of the food products 30.

Food monitors 32 are located on the cooler housing 22 adjacent the plurality of food products 30 with at least one food monitor 32 located adjacent one of the vents 28. The food monitors 32 can identify the food product 30 in the vicinity of the food monitor 32 and then monitor a condition of the food product 30.

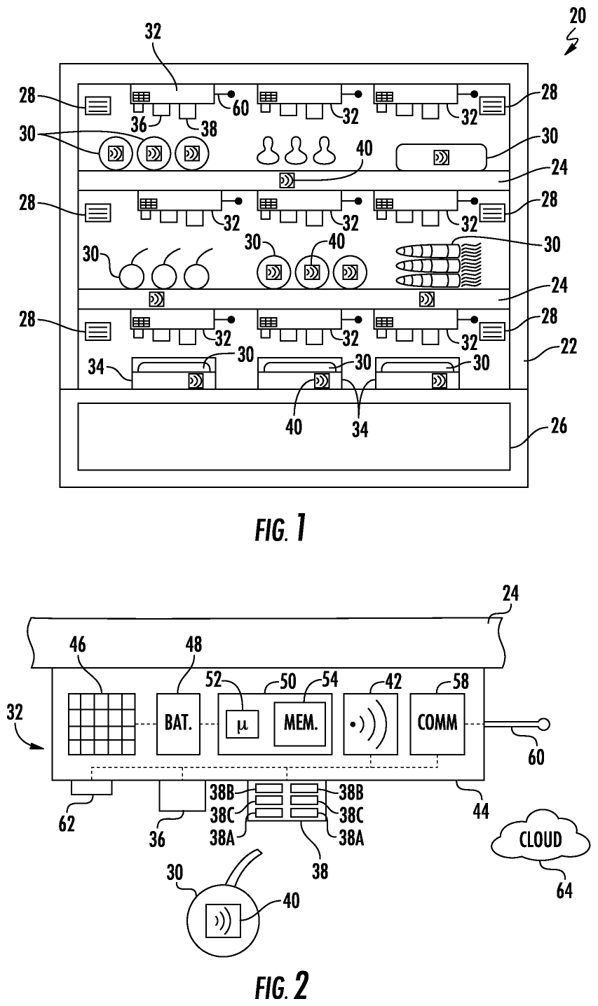

To identify the food product 30 in the vicinity of the food monitor 32, each of the food monitors 32 includes a RFID tag reader 42 (FIG. 2) for reading a RFID tag 40 associated with each individual food product 30 or type of food product 30. In one example, the RFID tag 40 is a sticker and is located directly on the individual food product 30. In another example, the RFID tag 40 is located on a container 34 storing the food product and in yet another example, the RFID tag 40 is located on the cooler housing 22 itself, such as on the shelf 24 displaying the food product 30. Although the illustrated example only shows three food monitors 32 associated with each shelf 24, the number of food monitors 32 associated with the refrigerated cooler 20 can vary depending on the number of different food products 30 stored in the refrigerated cooler 20 or the quantity of food products 30 stored in the refrigerated cooler 20.

FIG. 2 illustrates an enlarged view of the food monitor 32 located on an underside of the shelf 24 and adjacent the food product 30. In the illustrated example, the food product 30 includes the RFID tag 40 attached directly to the food product through the use of a sticker. The food monitor 32 is attached to an underside of the shelf 24. However, the food monitor 32 could be located on another portion of the cooler housing 22 of the refrigerated cooler 20 that provides the food monitor 32 an unobstructed view of the food product 30.

In the illustrated non-limiting example, the food monitor 32 includes a monitor housing 44 that is attached directed to the shelf 24. The monitor housing 44 includes a controller 50 having a microprocessor 52 and memory 54 for managing operation of the food monitor 32. The food monitor 32 can be powered in at least one of two ways. In one example, the food monitor 32 receives power directly from the refrigerated cooler 20. In another example, the food monitor 32 utilizes at least one solar cell 46 to harvest energy from the environment surrounding the refrigerated cooler 20. The energy generated from the at least one solar cell 46 can directly power the food monitor 32 or charge a battery 48 in electrical communication with the at least one solar cell 46. The battery 48 allows the food monitor 32 to continue operating when the at least one solar cell 46 is not generating enough power such as during night time operations.

The food monitor 32 also includes the RFID tag reader 42 in electrical communication with the controller 50 for identifying the food product 30. The controller 50 can access food product data stored in the memory 54 to determine an appropriate color range and pesticide level for the food product 30. The controller 50 can access data stored remotely, such as cloud data 64 stored in the cloud, through a communicator 58 having an antenna 60 through a Bluetooth or other wireless connection. Alternatively, the communicator 58 can be hard wired into a network for accessing the food product data stored remotely.

Once the food monitor 32 has identified the adjacent food product 30 and accessed the associated food product data, the food monitor 32 can measure a current color of the adjacent food product 30 with a color sensor 36. The color sensor 36 is configured to measure a wave length of light reflected off of the food product to determine the current color of the adjacent food product 30. The controller 50 can compare the current color of the food product 30 with a previously measured color of the food product 30 stored in the memory 54 or with an acceptable food product color range stored in the memory 54 to determine a condition of the food product 30. Alternatively, the controller 50 can compare the current color of the food product 30 with either a previously measured color of the food product stored in the cloud data 64 or an acceptable food product color range stored in the cloud.

The food monitor 32 also include a biosensor 38 for measuring gas emissions from the food products 30 and/or determining a pesticide level associated with the food product 30. The biosensor 38 includes at least one chemical sensor for measuring food product gas emissions and pesticide levels. The at least one chemical sensor includes a reactive bio-component element 38A, a sensor element 38B, and an interface element 38C disposed there between. The bio-component element 38A includes a bio-agent, such as bioactive species or biomimetic species, selected to interact specifically with a particular analyte to be sensed. The bio-agent, typically through a biochemical process, acts to bind or convert the analyte into a measurable component. The bio-component element 38A used in the illustrated example includes biological species such as enzymes, antigens, antibodies, receptors, tissues, whole cells, cell organelles, bacteria, and nucleic acids.

The sensor element 38B includes a physical component operative to generate a measurable output, usually an electrical or optical signal, indicative of the presence of the analyte and, in certain instances, the actual amount of the analyte that is received by the controller 50. The sensor element 38B includes at least one of an electrochemical device, an optical device, an acoustical device, or a calorimetric device.

The interface element 38C includes a membrane or coating that separates the sensor element 38B from the bio-component element 38A and serves as a link between the elements. In the illustrated example, the interface element 38C includes at least one of a polymer membrane, an electropolymerized coating, or a self-assembling monomers.

The biosensor 38 can monitor gas emissions from the food products 30 in the vicinity. Alternatively, the food monitor 32 can be located adjacent one of the vents 28 to determine if one of the food products 30 somewhere associated with the refrigerated cooler 20 is spoiling. The food monitor 32 sends a notification to a user regarding a possible food spoilage issue and directs the refrigeration system 26 to operate at a lower temperature to decrease the rate of spoilage or prevent the spoilage of the food products 30 from spreading. Alternatively, the food monitor 32 can illuminate a light 62 to indicate an undesirable condition of the food product 30. For example, the light 62 can illuminate red if risk of food spoilage is present or illuminate green if no risk is identified for spoilage.

It is also possible that the food products 30 may not be fit for consumption due to unacceptable levels of pesticides present with the food products 30. If the food monitor 32 determines that the food products 30 include an unacceptable level of pesticides, the food monitor 32 sends a notification to the user, such as a seller of the food products 30, of the possibility of pesticide contamination. The notification can be sent through the communicator 58 and the antenna 60 to the user or the food monitor 32 can illuminate the light 62 to indicate that the particular food product 30 is not fit for consumption. For example, the light 62 can illuminate red to indicate that the food product 30 is not acceptable for consumption or the light 62 can illuminate green to indicate that the food product 30 is acceptable for consumption.

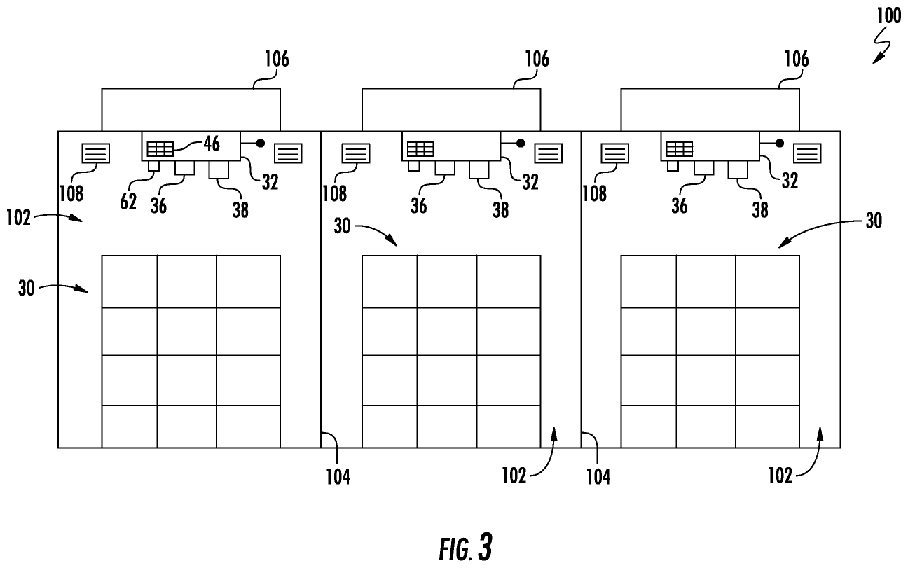

FIG. 3 illustrates an example refrigerated container 100 including multiple individual compartments 102 separated by divider walls 104 to allow the food products 30 to be stored at different temperatures. Each of the individual compartments 102 include at least one food monitor 32 that operates in a similar manner as the food monitors 32 described above with respect to the refrigerated cooler 20.

In particular, the food monitor 32 can use the color sensor 36 and biosensor 38 to monitor a condition of the food products 30 in each of the individual compartments 102 and communicate the condition of the food products 30 to the user as described above. However, because the individual compartments 102 rely on a greater degree of air circulation than the refrigerated cooler 20 above, positioning one of the food monitors 32 adjacent a vent 108 may be more effective in capturing the circulation of gas emissions from spoiling food. Additionally, the at least one solar cell 46 on the food monitor 32 may be less effective for the refrigerated container 100 because the individual compartments 102 for the refrigerated container 100 have less light exposure than the refrigerated cooler 20 described above.

The food monitors 32 in the refrigerated container 100 can work in conjunction with refrigeration systems 106 to adjust a temperature for the individual compartments 102. By adjusting the temperature for the individual compartments 102 separately and based on information obtained from the food monitors 32 regarding the condition of the food products 30 located within the individual compartments 102, the food products 30 stay fresh for longer periods of time, which reduces waste of the food products 30.

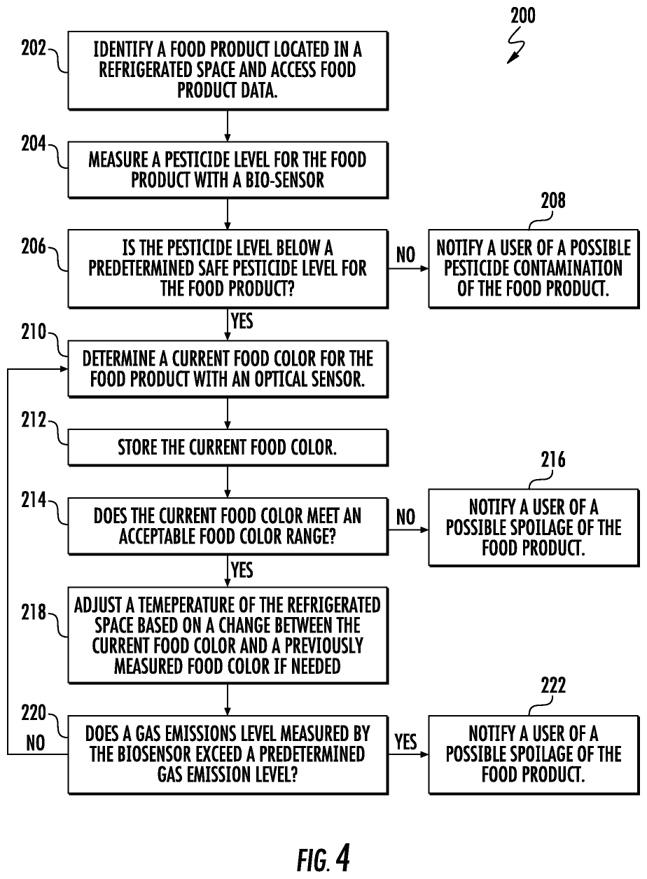

FIG. 4 illustrates a method of operating the food monitors 32 with either the refrigerated cooler 20 or the refrigerated container 100 to extend the freshness of the food products 30 and reduce waste. The food monitor 32 initially identifies the food product 30 located in the vicinity of the food monitor 32 through the use of the RFID tag reader 42. Block 202. Once the RFID tag reader 42 has identified the specific food product 30 associated with the RFID tag 40, the food monitor 32 is able to access food product data for the specific food product 30. The food product data can either be stored in the memory 54 in the food monitor 32 or accessed remotely by the food monitor 32, such as by accessing the cloud data 64.

Once the food monitor has identified the specific type of the food product 30 and accessed the associated food product data, the food monitor 32 measures a pesticide level associated with the food product 30 with the biosensor 38. Block 204. The food monitor 32 then determines if the measured pesticide level for the particular food product 30 is below a predetermined safe pesticide level for the food product 30. Block 206. If the measured pesticide level for the food product 30 exceeds the predetermined safe pesticide level, the food monitor 32 notifies the user of the possibility of pesticide contamination of the food product 30. Block 208.

If the pesticide level is below the predetermined safe pesticide level, the food monitor 32 will then determine a current food color for the food product 30 with the color sensor 36. Block 210. The current food color is then stored in at least one of the memory 54 or the cloud data 64 for analyzing changes in the food products 30. Block 212.

The food monitor 32 then determines if the current food color is within an acceptable food color range for the particular food product 30. Block 214. If the current food color is not within the acceptable food color range, the food monitor 32 notifies the user of the possibility of spoiled food products 30. Block 216.

The food monitor 32 then determines a change in color for the food product by comparing the current food color with a previously measured food color stored in the cloud data 64 or the memory 54. The food monitor 32 can communicate with the refrigeration system 26, 106 to adjust the temperature in the vicinity of the food products 30 to extend the life of the food product 30 if the change in color between the current food color and the previously measured food color is larger than a predetermined valve. Block 218.

The food monitor 32 also measures for the presence of a gas emission indicative of the food product 30 spoiling in the vicinity of the food monitor 32 and if the gas emission level exceeds a predetermined level. Block 220. If the measured gas emission level exceeds the predetermined level, the food monitor 32 notifies the user of the possibility of food product 30 spoilage. Block 222. The food monitor 32 can communicate with the refrigeration system 26, 106 to adjust the temperature in the vicinity of the food products 30 to extend the life of the food product 30 if the gas emission level exceeds the predetermined level.

If the biosensor 38 does not identify gas emissions beyond the acceptable emissions level, the method 200 returns to block 210 and will determine a new current food color for the food product 30 with the color sensor 36 and will repeat the above described comparisons to monitor for the food products 30 spoiling.

The preceding description is exemplary rather than limiting in nature. Variations and modifications to the disclosed examples may become apparent to those skilled in the art that do not necessarily depart from the essence of this disclosure. The scope of legal protection given to this disclosure can only be determined by studying the following claims.

What is claimed is:

1. A food monitoring assembly comprising:
at least one color sensor for measuring a color of a food product;
a RFID tag reader configured to identify a RFID tag in the vicinity of the RFID tag reader;
a controller in electrical communication with the color sensor and the RFID tag reader and configured to identify a type of the food product and determine if the color of the food product is within a predetermined color range; and
a communicator in electrical communication with the controller for communication with a remote location.

2. The assembly of claim 1, further comprising at least one biosensor in electrical communication with the controller.

3. The assembly of claim 2, wherein the at least one biosensor is configured to measure gas emissions emitted from the food product.

4. The assembly of claim 2, wherein the at least one biosensor is configured to measure a pesticide level associated with the food product.

5. The assembly of claim 2, wherein the at least one biosensor includes at least one chemical sensor having a reactive bio-component element, a sensor element, and an interface element disposed there between.

6. The assembly of claim 1, further comprising at least one solar cell in electrical communication with a battery for powering the assembly.

7. The assembly of claim 1, wherein the color sensor is configured to measure a wave length of light to determine the color of the food product.

8. The assembly of claim 1, wherein the food monitor assembly is located adjacent a refrigeration cooler.

9. The assembly of claim 1, wherein the food monitor assembly is located within a food transport trailer.

10. The assembly of claim 1, wherein the controller includes a microprocessor and memory for storing food product data.

11. The assembly of claim 1, wherein the communicator includes a wireless connection for communicating with the remote location.

12. A method of monitoring a condition of a food product comprising the steps of:
identifying a type of the food product with a RFID tag reader;
measuring a current color of the food product with at least one color sensor;
determining if the current color is within a predetermined range of colors for the food product; and
signaling to a user if the current color is outside of the predetermined range of colors for the food product.

13. The method of claim 12, further comprising measuring a pesticide level associated with the food product using at least one biosensor.

14. The method of claim 13, further comprising signaling to a user if the pesticide level is outside of a predetermined pesticide level range.

15. The method of claim 13, wherein the step of signaling to the user includes illuminating a light.

16. The method of claim 13, further comprising measuring a gas emissions level associated with the food product using the at least one biosensor.

17. The method of claim 16, further comprising signaling to the user if the gas emissions level is outside of a predetermined gas emissions range.

18. The method of claim 12, further comprising comparing the current color of the food product with a previously measured color of the food product to determine a color change of the food product.

19. The method of claim 18, further comprising adjusting a refrigerated system to reduce food spoilage if the color change of the food product exceeds a predetermined level.

20. The method of claim 12, further comprising accessing food product data for the food product after the type of food product has been identified.
